(19) United States
(12) Patent Application Publication
(10) Pub. No.: US 2008/0015596 A1
(43) Pub. Date: Jan. 17, 2008

(54) LARGE DIAMETER MULTIPLE PIECE BONE ANCHOR ASSEMBLY

(76) Inventor: Dale E. Whipple, East Taunton, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/741,128

(22) Filed: Apr. 27, 2007

Related U.S. Application Data
(60) Provisional application No. 60/796,057, filed on Apr. 28, 2006.

(51) Int. Cl. A61B 17/58

(52) U.S. Cl. 606/73; 606/62

(57) ABSTRACT

A bone anchor assembly is described having a large diameter for connecting a spinal connection element to bone. The assembly includes a receiver member for receiving the spinal connection element, a bone-engaging member and a core shaft for coupling the receiver member to the bone-engaging sleeve.

### CONTINUING DATA

This application claims the benefit of U.S. Provisional Patent Application Ser. No. 60/796,057, entitled “Large Diameter Multiple Piece Bone Anchor Assembly”, filed Apr. 28, 2006, which is hereby incorporated herein by reference.

### BACKGROUND

Spinal connection systems may be used in orthopedic surgery to align and/or fix a desired relationship between adjacent vertebrae. Such systems typically include a spinal connection element, such as a relatively rigid fixation rod, plate or dynamic connector, that is coupled to adjacent vertebrae by attaching the element to various anchoring devices, such as hooks, bolts, wires, or screws. The spinal connection element can have a predetermined contour that has been designed according to the properties of the target implantation site, and once installed, the spinal connection element holds the vertebrae in a desired spatial relationship, either until desired healing or spinal fusion has taken place, or for some longer period of time.

Spinal connection elements can be anchored to specific portions of the vertebra. Since each vertebra varies in shape and size, a variety of anchoring devices have been developed to facilitate engagement of a particular portion of the bone. Pedicle screw assemblies, for example, have a shape and size that is configured to engage pedicle bone. Such screws typically include a threaded shank that is adapted to be threaded into a vertebra, and a head portion having a spinal connection element receiving element, which, in spinal rod applications, is usually in the form of a U-shaped slot formed in the head for receiving the rod. A set-screw, plug, cap or similar type of closure mechanism, may be used to lock the rod into the rod-receiving portion of the pedicle screw. In use, the shank portion of each screw may be threaded into a vertebra, and once properly positioned, a fixation rod or dynamic connector may be seated through the rod-receiving portion of each screw and the rod or dynamic connector is locked in place by tightening a cap or similar type of closure mechanism to securely interconnect each screw and the connection element. Other anchoring devices also include hooks and other types of bone screws.

In certain procedures, such as those in the lumbar or sacral spine, it may be necessary to use a larger diameter pedicle screw capable of carrying large loads. A difficulty in using a larger diameter screw comes from the corresponding increase in the size of the receiver head to accommodate the larger diameter screw shank. The increased size of the head can interfere with the bony anatomy limiting the polyaxial range of motion of the screw head. Another problem associated with manufacturing a larger diameter top-loading screw is that the opening of the receiver member has to be larger to accept the large diameter screw shank, which creates the need for a larger closure mechanism. It is desirable to maintain the same size opening such that the same size closure mechanism may be used. Accordingly, a large diameter screw is needed that does not change the size of the closure mechanism.

### SUMMARY

Disclosed herein are embodiments of a bone anchor assembly having a large diameter shank. In one embodiment, the bone anchor assembly includes a receiver member having a recess for receiving a spinal connection element and a bore, a core shaft having a head and a distal end sized to extend through the bore, and a bone-engaging sleeve having a proximal end adapted to engage the distal end of the core shaft. In alternate embodiments, the head of the core shaft may be spherical and allow pivoting between the bone-engaging sleeve and the receiver member. The head of the core shaft may have a drive feature.

In an alternate embodiment the bone anchor assembly may include a bone engaging sleeve configured to engage bone, a receiver member for receiving a spinal connection element to be coupled to the bone engaging sleeve, the receiver member having a distal end having a bore sized to receive a core shaft, and a recess in communication with the bore, the recess being sized and shaped to receive the spinal connection element, and a core shaft adapted to pivotally couple the bone-engaging sleeve and the receiver member.

### BRIEF DESCRIPTION OF THE FIGURES

These and other features and advantages of the bone anchor assembly and methods disclosed herein will be more fully understood by reference to the following detailed description in conjunction with the attached drawings in which like reference numerals refer to like elements through the different views. The drawings illustrate principles of the bone anchor assembly and methods disclosed herein and, although not to scale, show relative dimensions.

FIG. 1A illustrates a cross-section of a large diameter bone anchor assembly.

FIG. 1B illustrates a side view of the bone anchor shown in FIG. 1A.

FIG. 1C illustrates an exploded view of the bone anchor shown in FIG. 1A.

FIG. 1D illustrates a cross-section of the bone anchor assembly including an exemplary spinal rod, compression member and closure mechanism.

FIG. 1E illustrates a perspective view of the bone anchor assembly of FIG. 1D.

FIG. 2A illustrates a perspective view of the core shaft of the bone anchor assembly shown in FIG. 1A.

FIG. 2B illustrates a cross-section of the core shaft shown in FIG. 2A.

FIG. 3A illustrates a perspective view of the bone-engaging sleeve of the bone anchor assembly shown in FIG. 1A.

FIG. 3B illustrates a cross-section of the bone-engaging sleeve shown in FIG. 3A.

FIG. 4 illustrates a cross-section of the core shaft and bone-engaging sleeve assembly.

FIG. 5A illustrates a perspective view of the compression member shown in FIG. 1D.

FIG. 5B illustrates a perspective view of the bottom of the compression member shown in FIG. 5A.

FIG. 5C illustrates a cross section of the compression member shown in FIG. 5B.

### DETAIL DESCRIPTION OF EXEMPLARY EMBODIMENTS

Certain exemplary embodiments will now be described to provide an overall understanding of the principles of the structure, function, manufacture, and use of the large diameter bone anchor assembly and methods disclosed herein. One or more examples of these embodiments are illustrated in the accompanying drawings. Those of ordinary skill in the art will understand that the large diameter bone anchor assembly and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The articles “a” and “an” are used herein to refer to one or to more than one (i.e. to at least one) of the grammatical object of the article. By way of example, “an element” means one element or more than one element.

The terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

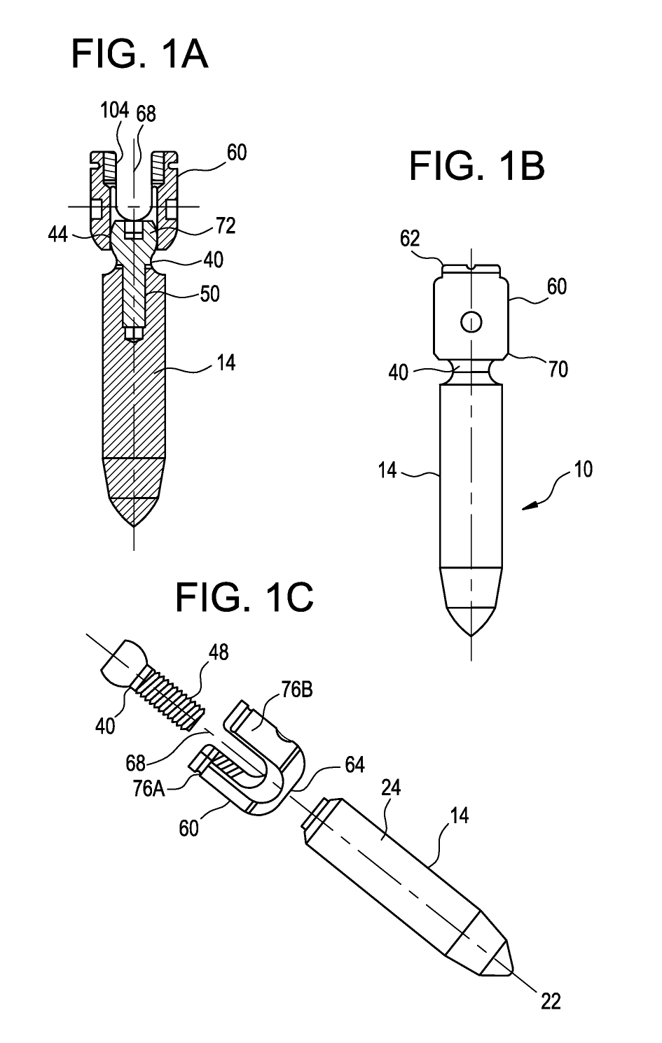

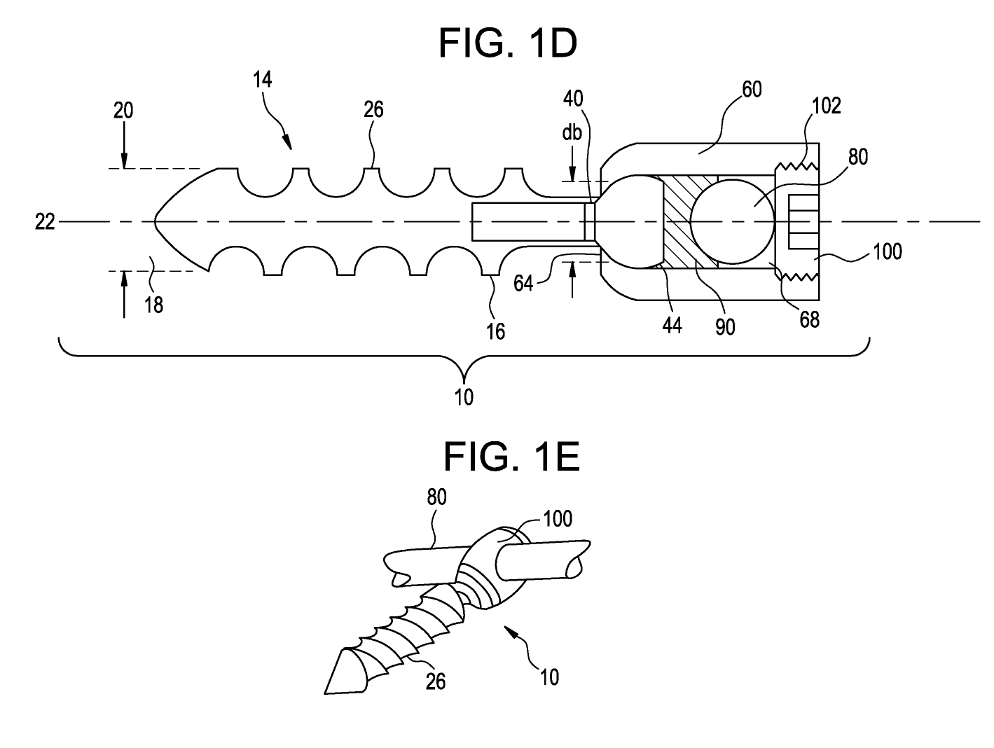

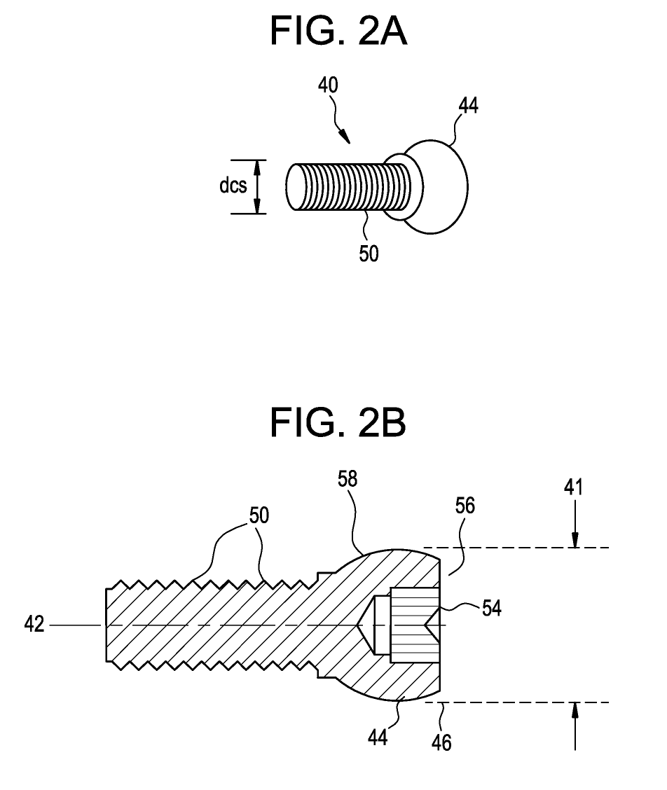

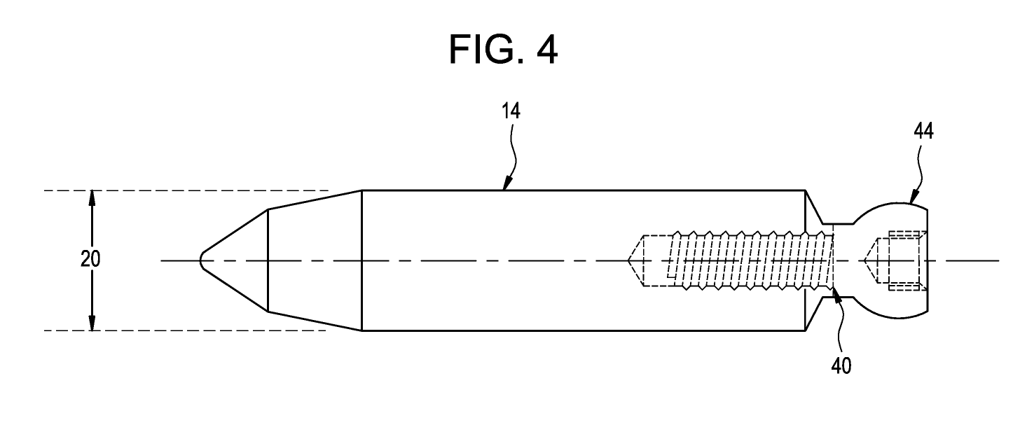

FIGS. 1-5 illustrate an exemplary embodiment of a large diameter bone anchor assembly. The exemplary bone anchor assembly 10 may be employed to engage one or more spinal connection elements to bone. For example, bone anchor assembly 10 may be employed to connect a spinal plate, rod (rigid or dynamic), and/or cable to a vertebra of the spine. Although the exemplary bone anchor assembly 10 described below is designed primarily for use in spinal applications, one skilled in the art will appreciate that the structure, features, and principles of the exemplary bone anchor assembly 10, as well as the other exemplary embodiments described below, may be employed to couple any type of orthopedic implant to any type of bone or tissue. Non-limiting examples of applications of the bone anchor assembly 10 described herein include long bone fracture fixation/stabilization, small bone stabilization, lumbar spine as well as thoracic stabilization/fusion, cervical spine compression/fixation, dynamic, non-fusion applications including facet replacement and dynamic posterior systems as well as skull fracture/reconstruction plating.

The illustrated exemplary bone anchor assembly 10 includes a bone-engaging member 14 configured for engaging bone, a receiver member 60 for receiving a spinal connection element, and a core shaft 40 for pivotally coupling the bone-engaging member 14 to the receiver member 60. The bone-engaging member 14 extends from a proximal end 16 to a distal end 18 along a longitudinal axis 22 and has a diameter 20. An outer surface 24 of the bone-engaging member 14 extends between the proximal end 16 and the distal end 18. The outer surface 24 of the bone-engaging member 14 may include one or more bone engagement mechanisms to facilitate gripping engagement of the bone anchor assembly 10 to bone. In the illustrated exemplary embodiment, for example, the bone-engaging member 14 includes an external thread 26 shown in FIGS. 1D and 1E. The external thread 26 may extend along at least a portion of the bone-engaging member 14. For example, in the illustrated exemplary embodiment, the external thread 26 extends from the distal end 18 to the proximal end 16 of the bone-engaging member 14. One skilled in the art will appreciate that bone engagement mechanisms other than external thread 26 may be employed, including, for example, one or more annular ridges, multiple threads, dual lead threads, variable pitched threads, and/or any other conventional bone engagement mechanism. In the illustrated exemplary embodiment, the diameter 20 of bone-engaging member 14 may be defined by the major diameter of external thread 26. The bone-engaging diameter 20 may be greater than the diameter 41 of the head 44 of the core shaft 40 described below.

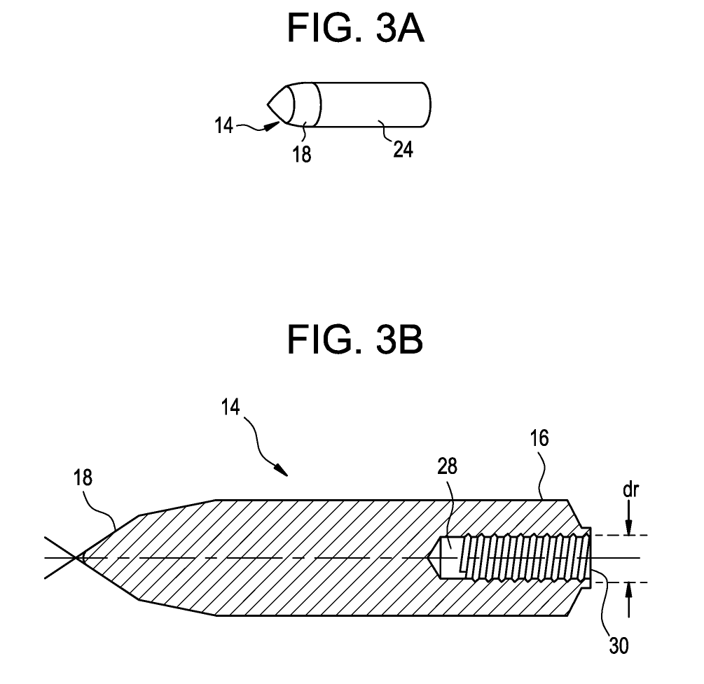

The proximal end 16 of the exemplary bone-engaging member 14 may be configured to receive the distal portion of the core shaft 40 of the bone anchor assembly 10 as described below. The proximal end 16 of the bone-engaging member 14 may have a recess 28 extending toward the distal end 18 along the longitudinal axis 22. The recess 28 has a diameter dᵣ. In the illustrated exemplary embodiment shown in FIG. 3B, for example, the recess 28 includes threads 30 extending from the proximal end 16 to the distal end of the recess 28. In an alternate embodiment, the recess 28 may be smooth.

The core shaft 40 of the bone anchor assembly 10, extends along the longitudinal axis 42 from a proximal end 46 to a distal end 48 and has a shank diameter d_{cs}. The distal end 48 of the core shaft 40 is sized to fit within the recess 28 of the bone-engaging member 14. The diameter of the core shaft d_{cs} is less than or equal to the diameter of the recess dᵣ. In the illustrated exemplary embodiment, for example the core shaft 40 may have external threads 50 extending along the distal end 48 to engage the threads 30 of the recess 28. In an alternate embodiment the core shaft 40 may be smooth.

The core shaft 40 has a head 44 at the proximal end 46 to facilitate adjustment of the bone-engaging member 14 relative to the receiving member 60 of the bone anchor assembly 10, as described below. For example, the head 44 may be approximately spherical in shape to permit pivoting of the bone-engaging member 14 relative to the receiving member 60. In the illustrated exemplary embodiment, for example, the head 44 may be in the shape of a truncated sphere having a generally planar proximal surface 56 and an approximately hemispherically shaped distal surface 58. The head 44 of the core shaft 40 may have surface texturing, knurling, and/or ridges. A drive feature 54 may be located internally or externally on the head 44 of the core shaft 40.

Referring to FIGS. 1A-E, the receiver member 60 of the exemplary bone anchor assembly 10 includes a proximal end 62 having a recess 68, and a distal end 70 having a bore 64. The receiver member 60, in certain exemplary embodiments, may be configured to receive a spinal connection element and couple the spinal connection element to the bone anchor assembly. In the exemplary embodiment, for example, the recess 68 of the receiver member 60 may be sized and shaped to receive a spinal rod 80, as illustrated in FIG. 1E. For example, the receiver member 60 has a generally U-shaped cross-section defined by two legs 76A and 76B separated by recess 68. Each leg 76A, 76B is free at the proximal end 62 of the receiver member 60. The exemplary spinal rod 80 may be seated within the recess 68 by aligning the spinal rod 80 and the recess 68, advancing the spinal rod 80 between the legs 76A and 76B into the recess 68. The configuration of recess 68 of the receiver member 60 may be varied to accommodate the type, size and shape of spinal connection element employed.

In the exemplary embodiment, the bore 64 of the receiver member 60 is sized to receive at least a portion of a bone anchor assembly, such as the core shaft 40 described above. For example, the distal end 48 of the core shaft 40 may extend through the bore 64, as illustrated in FIG. 1A. The diameter of the bore 64 is less than the diameter of the head 44 of the core shaft 40. The distal end 70 of the receiver member 60 may be sized and shaped to engage the head 44 of the core shaft 40. For example, the distal end 70 may define a seat 72 for engaging the head 44 of the core shaft 40 that allows the bone-engaging member 14 when assembled to the core shaft 40 to pivot relative to the receiver member 60. In some exemplary embodiments, the seat 72 may be approximately spherical in shape to permit pivoting of the bone-engaging member 14 relative to the receiver member 60. In the illustrated exemplary embodiment, the seat 72 may be approximately hemispherical in shape and may have a curvature analogous to the distal surface 58 of the head 44 of the core shaft 40. In other exemplary embodiments, the seat 72 may be tapered or may have any other shape that allows adjustment of the head 44 of the core shaft relative to the receiver member 60. In the exemplary embodiment, the bone anchor assembly 10 is a polyaxial bone anchor assembly. The bone-engaging member 14 when assembled to the core shaft may be pivoted to one or more angles relative to the receiver member 60.

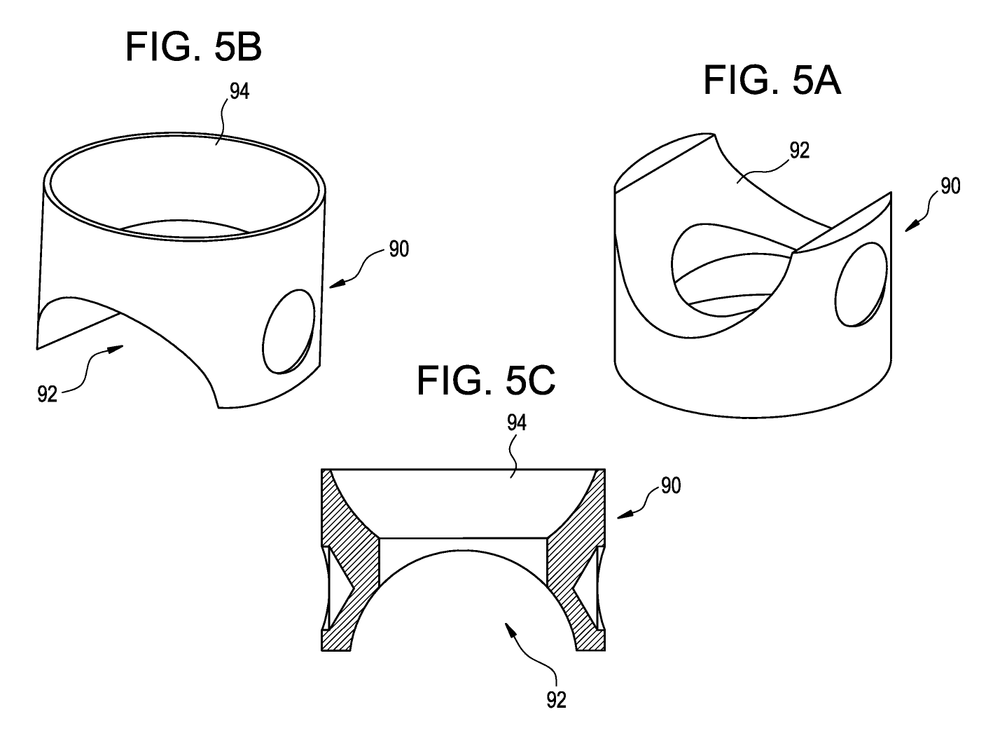

The bone anchor assembly 10 may optionally include a compression member 90 as shown in FIGS. 5A-C positionable within the receiver member 60 between the spinal connection element and the bone anchor. As illustrated in FIG. 1D, the compression member 90 may be positioned within the recess 68 between the spinal rod 80 and the head 44 of the core shaft 40. In the exemplary embodiment, the compression member 90 may have a proximal first surface 92 for engaging the spinal connection element and an opposing distal second surface 94 for engaging the head 44 of the core shaft 40.

The exemplary bone anchor assembly 10 may include a closure mechanism 100 that secures the spinal connection element to the bone anchor assembly. Referring to FIGS. 1D-E, the closure mechanism 100 secures the exemplary spinal rod 80 within the recess 68 of the receiving member 60. The closure mechanism 100 may engage the first end 62 of the receiving member 60 or, in other exemplary embodiments, may engage other portion(s) of the receiving member 60. The exemplary closure mechanism 100 is an internal set screw that engages an inner surface of the first end 62 of the receiving member 60. For example, the closure mechanism 100 may have external threads 102 that engage internal threads 104 provided on the first end 62 of the receiving member 60. Distal advancement of the closure mechanism 100 into engagement of the spinal rod 80, secures the spinal rod 80 within the recess 68 of the receiving member 60. In embodiments employing a compression member 90, such as exemplary bone anchor 10, distal advancement of the closure mechanism 100 into engagement with the spinal rod 80 seats the spinal rod 80 in the compression member 90. Distal advancement of the spinal rod 80 may also fix the bone-engaging member 14 relative to the receiving member 60 by engagement of the spinal rod 80 against the head 44 of the core shaft 40 or by engagement of the compression member 90 against the head 44 of the core shaft 40, as in the case of the illustrated exemplary embodiment.

One skilled in the art will appreciate that other types of closure mechanisms may be employed. For example, an external closure mechanism positionable around the outer surface of the legs 76A, 76B of the receiving member 60 may be employed. In other exemplary embodiments, the closure mechanism may comprise an external and an internal closure mechanism, a non-threaded twist-in cap, and/or any other conventional closure mechanism.

The components of the bone anchor assembly may be manufactured from any biocompatible material, including, for example, metals and metal alloys such as titanium and stainless steel, polymers, and/or ceramics. The components may be manufactured of the same or different materials. In one exemplary method of manufacturing, the bone-engaging member 14, the core shaft 40 and the receiver member 60 are separately constructed and assembled prior to implantation. The core shaft 40, in one exemplary method, may be coupled to the receiver member 60 by positioning the distal end 48 of the core shaft 40 through the bore 64 at the distal end 70 of the receiver member 60. The head 44 of the core shaft 40 may be seated within seat 72 such that the distal end 48 of the core shaft 40 extends through the bore 64. The compression member 90 may be positioned through the recess 68 of the receiver member 60 into engagement with the head 44 of the core shaft 40 before or after implantation.

The recess 28 of the bone-engaging member 14 receives the distal end 48 of the core shaft 40 to assemble the core shaft and the bone-engaging sleeve together while coupled with the receiver member 60. In one exemplary method, the distal end 48 of the core shaft 40 may engage threads 30 on the recess 28 of the bone-engaging sleeve to assemble the components together. In an alternate method, the distal end 48 of the core shaft 40 may frictionally engage or be press fit within the recess 28 of the bone-engaging member 14 to assemble the components while coupling the receiver member 60. After either of the above exemplary methods, the assembly may be pinned or welded together for additional security. Those of ordinary skill in the art will understand there are other methods of assembling the components together, including splining and clipping or swaging, or cinching.

While the large diameter multiple piece bone anchor assembly and methods of the present invention have been particularly shown and described with reference to the exemplary embodiments thereof, those of ordinary skill in the art will understand that various changes may be made in the form and details herein without departing from the spirit and scope of the present invention. Those of ordinary skill in the art will recognize or be able to ascertain many equivalents to the exemplary embodiments described specifically herein by using no more than routine experimentation. Such equivalents are intended to be encompassed by the scope of the present invention and the appended claims.

What is claimed:

1. A bone anchor assembly for engagement to a connection element comprising:
a receiver member having a recess for receiving the connection element and a bore;
a core shaft having a head and a distal end sized to extend through the bore of the receiver member; and
a bone-engaging member having a proximal end adapted to engage the distal end of the core shaft.

2. The bone anchor assembly of claim 1, wherein the head of the core shaft has a generally spherical shape.

3. The bone anchor assembly of claim 2, wherein the head of the core shaft has a drive feature.

4. The bone anchor assembly of claim 1, wherein the distal end of the core shaft is threaded.

5. The bone anchor assembly of claim 1, wherein the proximal end of the bone-engaging member has a recess.

6. The bone anchor assembly of claim 5, wherein the recess is threaded.

7. The bone anchor assembly of claim 1, wherein the bone-engaging member has an external thread.

8. The bone anchor assembly of claim 7, wherein the diameter of the bone-engaging member is greater than the diameter of the head of the core shaft.

9. The bone anchor assembly of claim 7, wherein the diameter of the bone engaging member is greater than the diameter of the bore of the receiver member.

10. The bone anchor assembly of claim 1, further comprising a compression member.

11. A bone anchor assembly comprising:
a bone engaging member configured to engage bone,
a receiver member for receiving a spinal connection element to be coupled to the bone engaging member, the receiver member having
a distal end having a bore, and
a recess in communication with the bore, the recess being sized and shaped to receive the spinal connection element, and
a core shaft adapted to pivotally couple the bone engaging member and the receiver member, a portion of the core shaft positionable within the bore of the receiving member.

12. The bone anchor assembly of claim 11, wherein the core shaft has a proximal end having an approximately spherical shaped head.

13. The bone anchor assembly of claim 11, wherein the core shaft has a threaded distal end.

14. The bone anchor assembly of claim 11, further comprising a compression member.

15. A bone anchor assembly for engaging a connection element comprising:
a receiver member adapted to receive a connection element, the receiver member having a distal end with a bore having a diameter;
a core shaft having a distal end adapted to extend through the bore of the receiver member and having a head with a diameter greater than the diameter of the bore; and
a bone-engaging member adapted to engage the distal end of the core shaft and having a diameter greater than the bore of the receiver member.

16. The bone anchor assembly of claim 15, wherein the head of the core shaft has an approximately spherical shape.

17. The bone anchor assembly of claim 15, wherein the head of the core shaft has a drive feature.

18. The bone anchor assembly of claim 15, wherein the bone engaging member has a recess at the proximal end for receiving the distal end of the core shaft.

19. The bone anchor assembly of claim 18, wherein the distal end of the core shaft engages the recess of the bone engaging member by a press-fit.

20. The bone anchor assembly of claim 18, wherein the recess of the bone engaging member is threaded.

21. The bone anchor assembly of claim 20, wherein the distal end of the core shaft is threaded.
